(19) United States
(12) Patent Application Publication
(10) Pub. No.: US 2026/0319175 A1
(43) Pub. Date: Oct. 15, 2026

(54) OPTICAL COMPONENT

(71) Applicant: UNIVERSITETET I TROMSØ - NORGES ARKTISKE UNIVERSITET, Tromsø (NO)

(72) Inventors: Jana Jágerská, Tromsø (NO); Marek Vlk, Tromsø (NO); Ganapathy Senthil Murugan, Tromsø (NO); Jehona Salaj, Tromsø (NO)

(21) Appl. No.: 19/109,279

(22) PCT Filed: Sep. 6, 2023
(86) PCT No.: PCT/EP2023/074502

§ 371 Date: Mar. 6, 2025

(30) Foreign Application Priority Data
Sep. 6, 2022 (GB) 2213015.7

(51) Int. Cl. G02B 6/125; G01N 21/3504; G02B 6/12; G02B 6/136

(52) U.S. Cl. CPC G02B 6/125; G01N 21/3504; G02B 6/136; G02B 2006/12138

(57) ABSTRACT

An optical component comprises a substrate, an input arranged to receive light, an output arranged to output light and a waveguide arrangement arranged to direct light from the input to the output, where the waveguide arrangement comprises a membrane extending over a channel in the substrate to form an unsupported portion that is separated from the substrate, where the unsupported portion comprises an uninterrupted core section extending between first and second surfaces of the membrane and a structured section adjacent the core section comprising a plurality of holes in said membrane.

### CROSS-REFERENCE TO RELATED APPLICATIONS

This application is a 35 U.S.C § 371 National Stage Entry of International Application No. PCT/EP2023/074502, filed Sep. 6, 2023, which claims the priority benefit of United Kingdom Application No. GB 2213015.7 filed Sep. 6, 2022, the contents of all of which are hereby incorporated herein by reference in their entireties for all purposes.

### TECHNICAL FIELD

The present invention relates to optical components such as those suitable for use in spectroscopy.

### BACKGROUND

A common application of spectroscopy is to measure absorption spectra of materials such as gases to identify and/or analyse the gas. A light beam is passed through a medium to be analysed, and the spectrum of the light once it has passed through is measured to identify absorption lines. The position, magnitude, width and/or shape of the absorption peaks can be analysed to deduce information about the medium, such as the presence and/or concentration of a particular chemical.

Traditional gas sensing with absorption spectroscopy uses large multi-pass cells containing a sample gas to be analysed. A light beam is reflected several times in the cell and its spectrum is analysed. Multi-pass cells are quite bulky and require large volumes of the sample gas.

More recently, chip-based sensors have been proposed in which light is directed through a long, coiled waveguide on a photonic chip whilst interacting with the surrounding gas. This can enable long interaction paths with a smaller overall sensor. However, current waveguides used in chip-based sensors achieve only relatively weak interactions with the surrounding medium (e.g. gas) and suffer from significant facet reflections which can reduce detection performance.

### SUMMARY

An improved approach may be desired.

According to a first aspect of the present invention there is provided an optical component comprising:
a substrate;
an input arranged to receive light;
an output arranged to output light; and
a waveguide arrangement arranged to direct light from the input to the output;
   wherein said waveguide arrangement comprises a membrane extending over a channel in the substrate to form an unsupported portion that is separated from the substrate, said unsupported portion comprising an uninterrupted core section extending between first and second surfaces of the membrane and a structured section adjacent the core section comprising a plurality of holes in said membrane; and
   wherein the core and structured sections form a waveguide arranged to direct light at least partway from the input to the output.

It will be recognised by those skilled in the art that, because the unsupported portion that is separated from the substrate, the waveguide formed by the core and structured sections is suspended within the surrounding medium (e.g. a gas to be analysed). In other words, when the optical component is oriented with the substrate underneath the membrane (which may be a normal operational orientation), the unsupported portion of the membrane may be suspended over a channel in the substrate so that the waveguide formed by the core and structured sections is free-standing, with the first and second surfaces in contact with the surrounding medium. Light propagating in the waveguide is partially confined by the interfaces at the first and second surfaces between the core section and the surrounding medium, and is partially confined by the structured section adjacent the core section.

Light propagating in the waveguide formed by the core section can become strongly delocalised outside the first and second surfaces of the membrane, such that the evanescent field of the light interacts extremely strongly with the surrounding medium, improving the performance of a sensing system using the optical component (e.g. improving an accuracy with which the depth and position of absorption maxima can be measured). In fact, the optical component may be arranged such that the interaction of an evanescent field of light propagating in the waveguide with a surrounding medium exceeds an interaction possible with an equivalent length free-space beam (as a consequence of the combined effect of mode delocalization and waveguide dispersion). The light interacts weakly with the core section, minimising propagation loss due to scattering or material absorption and minimising facet reflections associated with some conventional waveguides (i.e. minimising undesired etalon effects in a spectroscopy system using the optical component). Minimising facet reflections may also reduce back-reflections into a light source providing light to the input (e.g. a laser), which can negatively affect the light source (e.g. impacting laser tuning). The optical component can thus provide good sensing capabilities whilst having a smaller size and weight and requiring less of the sample to be analysed compared to conventional approaches (e.g. multi-pass cells).

The structured section adjacent the core section acts as a microstructured cladding layer on at least one side of the waveguide, providing at least partial lateral confinement of light travelling in the waveguide. The structured section may extend from the first or second surface and preferably extends between the first and second surfaces, i.e. so as to provide cladding along the entire depth of the core section. The presence of the holes causes the structured section to act as an effective medium of low refractive index, preventing light from propagating outside of the core section whilst simultaneously facilitating distribution of the surrounding medium (e.g. gas to be analysed) to both sides of the membrane. Furthermore, as explained in more detail below, the holes can also facilitate manufacture of the optical component by enabling under-etching of the substrate through the membrane.

As explained above, the unsupported section is separated from the substrate by a distance defined by the channel and sufficient to allow a strong interaction between the evanescent field of light propagating in the waveguide and the surrounding medium, without interacting with the substrate. The unsupported section may be separated from the substrate by 10 μm or more, 20 μm or more or 50 μm or more.

Some parts of the unsupported portion (e.g. the core section) may be further from the substrate than other parts (e.g. the structured section), e.g. due to the method used to form the channel. In such cases the closest part of the unsupported section may be separated from the substrate by 10 μm or more, 20 μm or more or 50 μm or more.

The unsupported portion of the membrane may extend from at least one portion of the membrane that is supported by the substrate, e.g. directly by the substrate, without any intermediate layers. For instance, a portion of the membrane adjacent the unsupported portion may be connected (e.g. directly) to the substrate, with the unsupported portion extending from the connected, supported portion as a cantilever. Alternatively, the unsupported portion may extend between two or more supported portions. In some embodiments, there may be one or more intermediate layers between the supported portion(s) of the membrane and the substrate (e.g. the membrane may be connected to the substrate via one or more intermediate layers). In such embodiments the intermediate layers do not extend between the unsupported portion and the channel in substrate. In a set of embodiments, the one or more supported portions are connected to the substrate adjacent the channel.

The core section of the membrane may extend from a first boundary to a second boundary in a direction transverse to the extension of the waveguide arrangement, with the structured section adjacent at least part of either or both of the first and second boundaries, i.e. with the structured section defining the boundary(ies) of the core section. Preferably, the structured section laterally surrounds the core section in (i.e. it is adjacent both of the first and second boundaries). In such embodiments, the structured section effectively comprises two parts, with one on each side of the core section (i.e. providing a cladding layer on two laterally opposite sides of the waveguide). The structured section may provide cladding along the entire core section.

The waveguide arrangement may be arranged to support any suitable propagation mode. However, in a set of embodiments the waveguide arrangement may be arranged to direct light propagating in a transverse magnetic (TM) mode. The waveguide arrangement may be arranged to direct light of many different wavelengths from the input to the output. In a set of embodiments, the waveguide arrangement is arranged to direct infrared light from the input to the output, e.g. light having a wavelength between 0.5 μm and 100 μm. In a set of embodiments, the waveguide arrangement is arranged to direct mid-infrared light from the input to the output, such as light having a wavelength between 2 μm and 20 μm. The waveguide arrangement may be arranged to direct a range of wavelengths of light, e.g. light between 2 μm and 3 μm, or light between 3.4 μm and 4.5 μm, e.g. light having a wavelength of approximately 4.35 μm for sensitive detection of CO₂ isotopes. The optical component may be arranged for use in mid-infrared spectroscopy.

The membrane may extend parallel to the substrate. For instance, the substrate and the membrane may each be substantially planar and extend in parallel planes. The membrane may be a thin membrane, i.e. with a thickness between the first and second surfaces that is less and preferably much less than a lateral width of the membrane. The membrane may be of substantially constant thickness. The membrane may comprise a thickness of 1 μm or less, 750 nm or less, 500 nm or less, 400 nm or less, 350 nm or less, 250 nm or less or 100 nm or less. It will be appreciated that because the core section extends between the first and second surfaces, the thickness of the membrane defines the thickness of the waveguide formed by the core and structured sections. In a set of embodiments, the membrane comprises a thickness between 50 nm and 1000 nm, e.g. between 200 nm and 650 nm, between 400 nm and 650 nm or between 500 nm and 650 nm. These ranges of thicknesses may be particularly suitable for mid-IR operation, e.g. directing light with wavelengths around 4.35 μm, for CO2 detection. In a set of embodiments, the thickness of the membrane is selected by maximizing the product of electric energy fraction in the gaseous environment and the waveguide group index. For instance, the product of electric energy fraction and the waveguide group index may define a so-called confinement factor, which determines the sensitivity of interaction of the guided light with the surrounding environment (external confinement factor) or the core (internal confinement factor). In a set of embodiments, the thickness of the membrane is selected to maximize an external confinement factor (e.g. to maximize the sensitivity to absorption in the surrounding gas), and/or to minimize an internal confinement factor (e.g. to minimize the absorption loss or scattering in the core section) of the waveguide formed by the core and structured sections. The material and other parameters (e.g. width, shape) of the core and structures sections may also, or alternatively, be selected based on a desired confinement factor (e.g. to minimise or maximise an internal or external confinement factor).

The waveguide formed by the core and structured sections may have a length (i.e. in the direction in which it directs light) of 1 mm or more, 5 mm or more, 10 mm or more, 25 mm or more or even 100 mm or more. The waveguide may be linear, but preferably the waveguide follows a curved path (e.g. in a plane parallel to the substrate), as this can provide a larger interaction length for a given overall size of the optical component. In a set of embodiments, the waveguide follows a path that contains one or more complete loops (i.e. a path that passes through 360°) between the input and the output. In a set of embodiments, the waveguide follows a clothoid curve path in which the curvature and the first derivative of curvature (i.e. the change of curvature) are minimised. The whole length of the waveguide may follow a clothoid curve. The use of a clothoid curve path may help to minimize radiation losses associated with large curvatures and/or transition losses associated with abrupt curvatures changes.

The waveguide formed by the core and structured sections may have a width (i.e. in a lateral direction transverse to the direction in which it directs light) selected to support single-mode operation for a given membrane (e.g. with a given material and/or dimensions) and a given operating wavelength (or range of wavelengths).

The waveguide formed by the core and structured sections may have a width of 1 μm or more, 5 μm or more, 10 μm or more or 20 μm or more. The waveguide may have a width of 50 μm or less, 25 μm or less or 10 μm or less. The unsupported portion of the membrane may have a width of 500 μm or less, 250 μm or less or 150 μm or less. The unsupported portion of the membrane may have a width of 10 μm or more, 20 μm or more, 50 μm or more or 100 μm or more. In a set of embodiments the unsupported portion of the membrane has a width of approximately 120 μm.

The optical component may comprise a maximum dimension of 50 mm or less, 25 mm or less, or 10 mm or less. The optical component may have a height (i.e. a direction perpendicular to the first and/or second surface) between 50 μm and 5 mm, e.g. between 100 μm and 2 mm or between 250 μm and 1 mm. The optical component may comprise a photonic chip. The optical component may be arranged to be used directly with a surrounding environment (i.e. without a chamber containing a specific sample to be analysed). Alternatively, the optical component may be arranged to be used with an adjacent sample chamber containing a sample to be analysed, wherein the unsupported portion of the membrane is exposed to the sample to be analysed. The optical component may comprise an integral sample chamber adjacent the unsupported portion of the membrane.

The waveguide arrangement may comprise several different waveguides (including the waveguide formed by the membrane) connected in series to direct light from the input to the output. However, in a set of embodiments the waveguide formed by the core and structured sections directs light all the way from the input to the output. The input and/or the output may preferably comprise an end of the core section of the membrane.

The optical component may have only a single waveguide arrangement extending from a single input/output pair. However, in a set of embodiments the optical component comprises a plurality of parallel waveguide arrangements, e.g. for guiding different frequencies of light simultaneously. Some or all of the parallel waveguide arrangements may extend from a common input and/or to a common output. In some embodiments one or more of the parallel waveguide arrangements has a dedicated input and/or output. For instance, a plurality of parallel waveguide arrangements may extend from an array of separate inputs to an array of separate outputs.

In a set of embodiments, the first and/or second surfaces of the membrane are substantially planar, i.e. substantially free of substantial and/or discontinuous projections or impressions. In such embodiments, where the structured section extends from the first or second surface (or extends between the first and second surfaces), the core section and the structured section define a common plane at the respective surface(s). In other words, the core section may not protrude relative to the structured section (or vice-versa) at the first and/or second surface. For instance, the core section may not comprise a rib or rib-like structure that protrudes relative to the structured section. Some manufacturing techniques may result in part or all of the structured section being slightly thinner than the core section (e.g. 5-15% thinner). However, it will be appreciated that such thinning has no meaningful impact on waveguide operation and in such examples the core section and the structured section are still considered to define a common plane at the first and/or second surface.

In a set of embodiments, the core and structured sections are formed from a single monolithic membrane, i.e. the core and structure sections are not manufactured separately and then connected together. The core and/or the structured sections may consist of a single material, i.e. with neither the core nor the structured section comprising additional materials or layers. This may simplify manufacture.

In a set of embodiments, the membrane is formed from Silicon Nitride (SiNₓ), Tantalum Oxide (Ta₂O₅), Aluminium Oxide (Al₂O₃), Silicon (Si), Germanium (Ge), Diamond (C), Indium Phosphide (InP), or Gallium Arsenide (GaAs). The substrate may be formed from Silicon.

As mentioned above, using a structured section with holes to confine the light laterally in the central portion may reduce losses, in particular those from TM/TE mode cross-coupling or material absorption. When implemented appropriately, the waveguide formed by the core and structured sections may direct light substantially or entirely losslessly. This allows the use of long waveguide structures and correspondingly long interaction lengths with the surrounding medium, improving the performance of sensing systems using the optical component (e.g. spectrometry systems).

The holes of the structured section may have the same or different geometrical shapes and sizes. The holes may have a maximum dimension of 1.0 μm or less, 1.6 μm or less, 2 μm or less or 5 μm or less. In a set of embodiments, some or all of the holes have partial or full rotational symmetry about an axis parallel to their extension through the membrane (i.e. perpendicular to a planar membrane). For instance, the holes may be substantially circular.

At least some of the holes in the membrane must be through-holes, i.e. extending all the way through the membrane. However, one or more of the holes may not extend all of the way through the membrane whilst still providing confinement effects.

In a set of embodiments, some of or all of the holes in the structured section form a periodic array such as a regular or irregular lattice. For instance, the holes may form a triangular or rectangular lattice. Additionally or alternatively, the holes may form a rectangular grating or another geometric pattern. The structured section may comprise a photonic crystal.

One or more parameters of the holes may be engineered to optimise confinement and minimise losses. Optimisable parameters include the size of holes, their average spacing, a period of a lattice of holes, a filling factor of holes (i.e. a fraction of the area of the structured section occupied by holes), and a density of holes. In a set of embodiments, one or more parameters of the holes is/are chosen such that a TM guided mode in the core section having a given frequency falls below a dielectric band defined by the holes in the structured section.

In a set of embodiments, the holes may have an average spacing that is less a target wavelength of light for which the waveguide arrangement is arranged, and preferably less than half said target wavelength. The holes of the structured section may have a density (i.e. a number density of holes per unit area of the structured section sufficiently high to prevent light of a target frequency range from propagating outside of the central portion. In a set of embodiments, the structured section is arranged such that any line drawn through the structured section in a direction transverse to the extension of the waveguide arrangement passes through at least one hole and preferably through at least two holes. It may be important to ensure that the density of the holes is not too high, to avoid structurally weakening the unsupported portion of the membrane. It is also desirable to use a density of holes that is manufacturable using standard lithography techniques.

In a set of embodiments, a dimension of the holes (e.g. a radius of circular holes) is selected to be the maximum possible without comprising mechanical stability. This may maximize lateral mode confinement in the core region and thus minimize any bending losses in curved portions of the waveguide.

For example, a waveguide arrangement designed for CO₂ sensing (e.g. with light having a wavelength around 4.35 μm), the structured section may comprise a triangular lattice of circular holes, with a period of 2 μm and a hole diameter of 1.6 μm, in a SiNx membrane that is 500 nm thick.

As mentioned above, the optical component may be particularly useful for use in sensing applications. Accordingly, the invention extends to a sensing system comprising:
a light source;
the optical component as disclosed herein, with the input arranged to receive light from the light source; and
a sample volume adjacent the optical component;
a detector arranged to receive light from the output of the optical component.

The spectroscopic sensing system may be used simply to sense the environment surrounding the optical component (i.e. the sample volume may simply comprise an adjacent section of the surrounding environment). However, in a set of embodiments, the spectroscopic sensing system includes a sample chamber adjacent the optical component for holding a (e.g. gaseous or liquid) sample to be sensed and defining the sample volume. As mentioned above, the optical component disclosed herein may support good sensitivity over conventional systems using only small sample volumes. The sample volume may have a volume of 100 μl or less, 50 μl or less, 10 μl or less or even 5 μl or less. This may be compared to necessary sample volumes of millilitres or litres for free-space optics-based instruments.

The light source may be arranged to produce monochromatic light (i.e. light predominantly having a single wavelength). For instance, the light source may comprise a laser. The wavelength of light produced by the light source may be controllable, e.g. within a particular range of interest.

The detector may be arranged to sense spectra of light received from the output of the optical component. In other words, the system may comprise a spectroscopy sensing system (e.g. for IR laser absorption spectroscopy). The detector may be arranged to measure an intensity of light received from the output of the optical component. The detector may be arranged to measure one intensity at a time, e.g. as the wavelength of the input light is swept over a target range. Alternatively, the detector may be operable to measure a plurality of intensities of different wavelengths of light simultaneously, i.e. with the light source providing light comprising a mixture of wavelengths.

The sensing system may comprise a pump arranged to lower a pressure in the sample volume, e.g. to reduce the width of absorption peaks. The pump may be integrated with or separated to the optical component.

When viewed from a second aspect of the present invention there is provided a method of manufacturing an optical component comprising:
applying a membrane to a substrate; and
forming a waveguide arrangement using said membrane and said substrate, said waveguide arrangement arranged to direct light from an input arranged to receive light to an output arranged to output light;
wherein forming said waveguide arrangement comprises:
   forming a plurality of holes in the membrane to form a structured section adjacent an uninterrupted core section extending between first and second surfaces of the membrane;
   under-etching the substrate through the holes in the membrane to form a channel in the substrate aligned with the structured section and the uninterrupted core section, such that the core and structured sections are comprised by an unsupported portion of the membrane that is separated from the substrate and such that the core and structured sections form a waveguide arranged to direct light at least partway from the input to the output.

It will be appreciated by those skilled in the art that the core and structured sections effectively being formed in a single step of forming holes in the membrane may facilitate low cost manufacture of long, high performance waveguides. As explained above, using a structured section comprising a plurality of holes for lateral confinement may allow the optical component to provide improved sensing capabilities. Under-etching the substrate through the holes to form the unsupported portion of the membrane (i.e. to remove support from part of the membrane) may also be relatively straightforward compared to other possible methods for suspending the membrane.

Forming the plurality of holes may comprise one or more standard lithography techniques, that may use a photomask or be maskless, such as UV photolithography, Deep-UV photolithography, Extreme UV photolithography and Electron beam lithography.

Under-etching the substrate may comprise molecular gas etching (e.g. with XeF₂) and/or reactive ion etching (e.g. with SF₆).

Features of any aspect or embodiment described herein may, wherever appropriate, be applied to any other aspect or embodiment described herein.

Where reference is made to different embodiments, it should be understood that these are not necessarily distinct but may overlap.

### BRIEF DESCRIPTION OF THE DRAWINGS

One or more non-limiting examples will now be described, by way of example only, and with reference to the accompanying figures in which:

FIG. 1 is a schematic diagram of a spectroscopic sensing system according to an embodiment of the invention;

FIG. 2 is a side view of the spectroscopic sensing system;

FIG. 3 is a cutaway view of the waveguide arrangement of the spectroscopic sensing system;

FIG. 4 is a side view of the waveguide arrangement;

FIGS. 5-7 illustrate various steps in the manufacture of the spectroscopic sensing system; and FIGS. 8 and 9 are band diagrams of possible waveguide arrangements.

### DETAILED DESCRIPTION

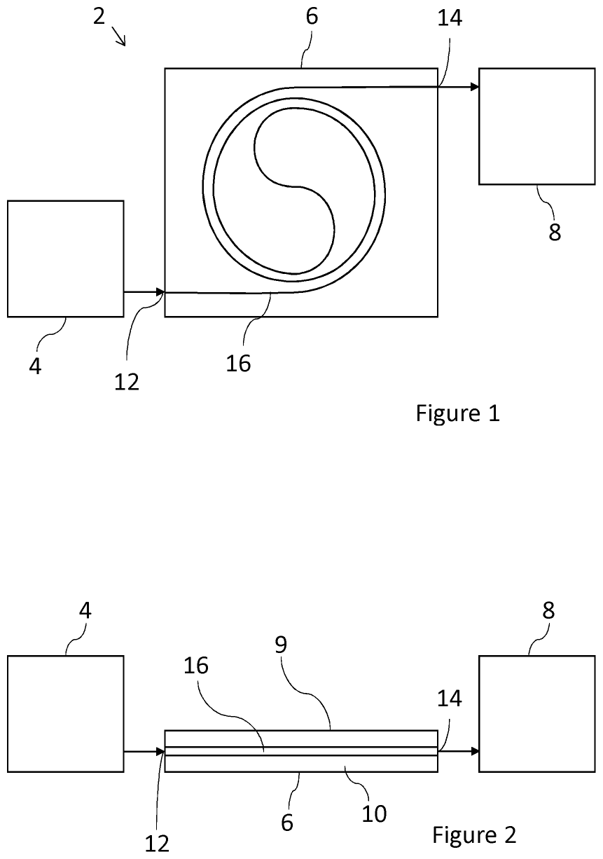

FIGS. 1 and 2 show a spectroscopic sensing system 2 for analysing the composition of a gas. The system 2 comprises a laser light source 4, a photonic chip 6, a detector 8 and a sample chamber 9. FIG. 2 shows the spectroscopic sensing system 2 in a typical operational orientation in which the sample chamber 9 is directly on top of the photonic chip 6.

The photonic chip 6 comprises a substrate 10, an input 12 for receiving input light from the laser 4, an output 14 for outputting light to the detector 8 and a waveguide arrangement 16 that extends from the input 12 to the output 14 to guide light from the input 12 to the output 14.

The waveguide arrangement 16 extends along a curved path with several loops to increase the length over which the light travelling in the waveguide arrangement 16 interacts with a sample in the sample chamber 9, without increasing the size of the photonic chip 6.

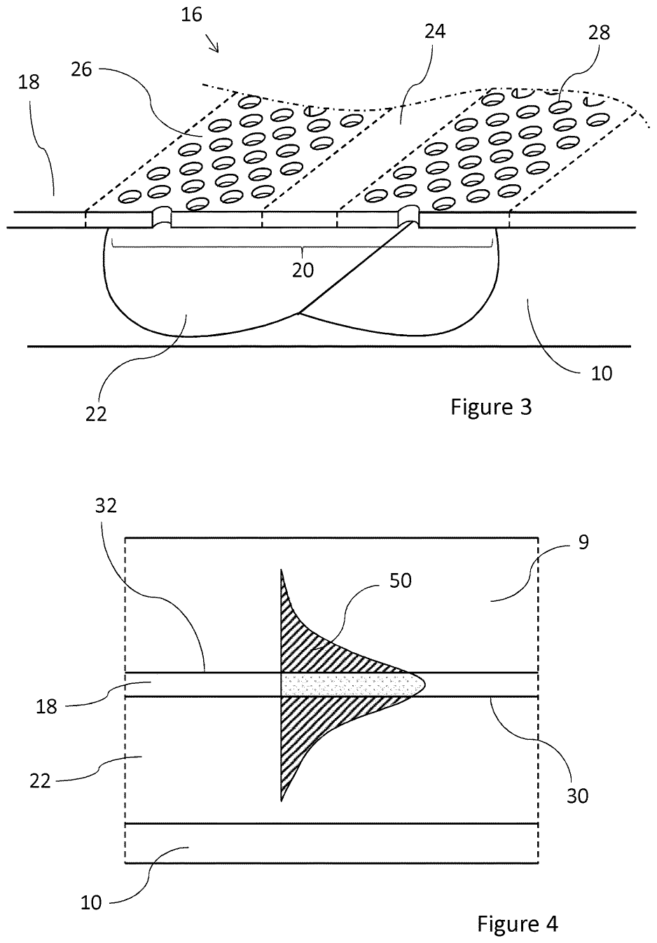

As shown in FIGS. 3 and 4, the waveguide arrangement 16 comprises a thin membrane 18 on top of substrate 10. The membrane 18 has an unsupported portion 20 which extends over a channel 22 in the substrate 10 and is thus separated from the substrate 10. The unsupported portion 20 comprises an uninterrupted core section 24 and a structured section 26 comprising a lattice of holes 28 in the membrane 18. The structured section 26 is split into two parts, with one part adjacent each side of the core section 24.

The uninterrupted core section 24 and the structured section 26 together form a waveguide that directs light from the input 12 to the output 14. The structured section 26 borders the core section 24 on either side transverse to the extension of the waveguide arrangement 16 and acts as a cladding layer on either side of the core section 24, providing lateral confinement of light travelling in the waveguide arrangement 16. In the orientation illustrated in the Figures, the core section 24 and the structured section 26 extend from a bottom surface 30 of the membrane 18 to a top surface 32 of the membrane 18.

In use, a gas to be analysed is introduced to the sample chamber 9. The gas spreads through the holes 28 into the channel 22 below the membrane 18.

The laser 4 produces monochromatic light at a first wavelength. The light is received at the input 12 and directed through the waveguide arrangement 16 to the output 14. As shown in FIG. 4, because the core section 24 is formed in the unsupported portion 20 of a thin membrane 18, the evanescent field 50 of light travelling in the core section 24 interacts very strongly with the surrounding gas.

The interaction of the evanescent field 50 may even exceed the interaction possible with an equivalent length free-space beam (e.g. up to 107% of the interaction). The period of the lattice is chosen as explained below with reference to FIGS. 8 and 9 to ensure that light is laterally confined in the core section 24 and to enable lossless propagation of light through the waveguide arrangement 16.

The detector 8 records the intensity of light output from the output 14. The process repeats, with the laser 4 sweeping through a range of wavelengths, to build up an intensity spectrum of light passing through the waveguide arrangement 16 and interacting with the sample gas. The location and size of peaks and troughs in the spectrum can be analysed to determine information about the composition of the sample gas using normal spectroscopic techniques.

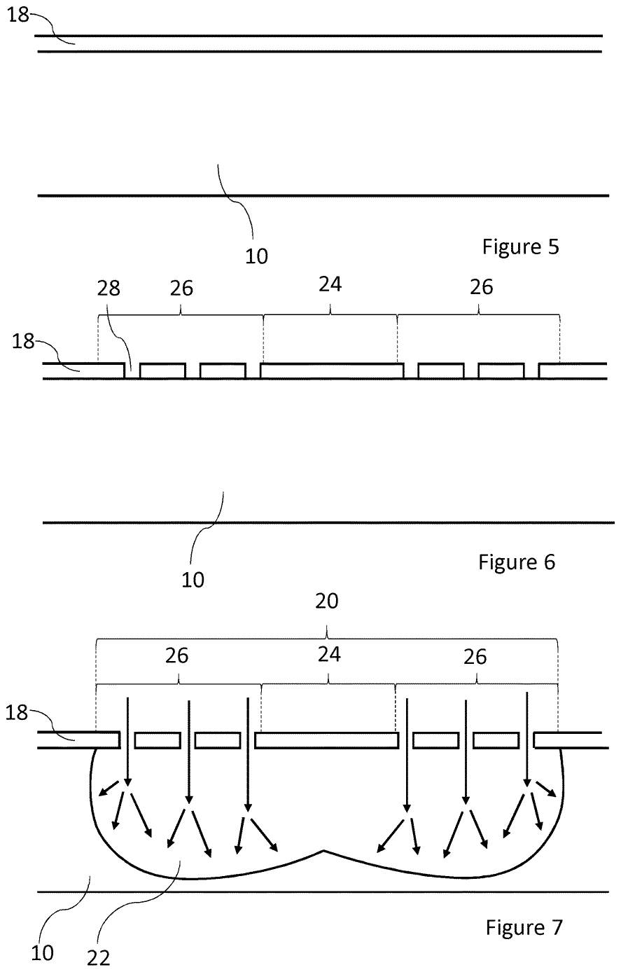

FIGS. 5-7 illustrate various steps in a method of manufacturing the photonic chip 6. In a first step, shown in FIG. 5, the membrane 18 is applied to the substrate 10.

In a second step, shown in FIG. 6, a plurality of holes 28 are formed in the membrane 18 using standard lithographic techniques. The holes 28 form two parts of a structured section 26 on either side of an uninterrupted core section 24. The uninterrupted core section 24 is effectively defined by the placement of the holes 28. The holes 28 are positioned so that the structured section 26 and the uninterrupted core section 24 follow a desired waveguide path along the substrate 10 from an input for receiving light (e.g. an end facet of the membrane at an edge of the substrate) to an output for outputting light (e.g. another end facet of the membrane at an edge of the substrate)

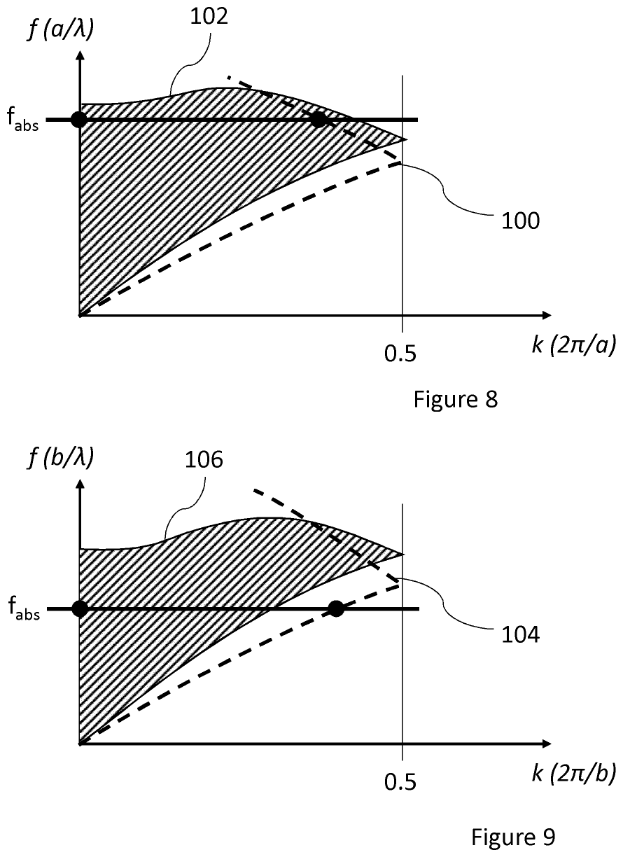

In a third step, shown in FIG. 7, a part of the substrate 10 aligned with the structured section 26 and the uninterrupted core section 24 (underneath the structured and core sections in the orientation used in FIG. 7) is removed by under-etching through the holes 28. This forms a channel 22 in the substrate, with an unsupported portion 20 of the membrane 18 containing the structured and core sections 26, 24 extending over the channel 22.

The under-etching process leaves the unsupported portion 20 suspended within the sample to be tested, so that (as explained above) the evanescent field of the light travelling in the core section 24 interacts extremely strongly a sample located above and below the membrane 18 (e.g. in the sample chamber 9 and in the channel 22, having spread through the holes 28 from the sample chamber 9), improving the performance of the sensing system 2.

The selection of the period of the lattice will now be described with refence to FIGS. 8 and 9, which illustrate the relationship between the line-defect waveguide mode (for the single-mode case) propagating in the core section 24 and the dielectric band of the lattice of holes 28 in frequency (f)—wave vector (k) space. The lattice of holes 28 effectively acts as a photonic crystal mirror on either side of the core section 24.

The operation frequency f_{abs} of the sensing system 2, which is defined by the absorption line(s) of interest in the gas sample to be analysed. For example, for sensing CO₂ the operation frequency f_{abs} is 2301 cm⁻¹ (i.e. λ=4.34 μm).

FIG. 8 illustrates the line-defect waveguide mode 100 and a dielectric band 102 of the structured section 26 when the lattice of holes 28 has a first period, a. The line-defect waveguide mode 100 is an increasing function of k towards k=0.5, where it gets “folded back” by the photonic crystal lattice of holes 28. The dielectric band 102 represents a subset of the f-k space where extended states exist in the photonic crystal lattice of holes 28.

It can be seen for the lattice period illustrated in FIG. 8, the waveguide mode 100 at the operation frequency f_{abs} is within the dielectric band area 102. As such, light propagating in this mode can propagate in the structured section 26. This is therefore a non-optimal “leaky” channel for the waveguide mode 100.

FIG. 9 illustrates the line-defect waveguide mode 104 and a dielectric band 106 of the structured section 26 when the lattice of holes 28 has a second, smaller period b (i.e. a higher density of holes 28 in the structured section 26). In this scenario, the waveguide mode 104 at the operation frequency f_{abs} is outside the dielectric band area 106. The mode 104 can propagate losslessly (a “guided” mode”).

Preferably, the period is chosen such that the waveguide mode 104 at the operation frequency f_{abs} is well below the folding of the mode, because in the near proximity of folding frequency the losses of the mode can increase due to localization, and above the folding frequency the dielectric band may be very close, with only a slight variation in the photonic lattice parameters required to make the mode and band overlap. As mentioned above, the selection of the period also takes into account structural and manufacturing limits.

While the invention has been described in detail in connection with only a limited number of embodiments, it should be readily understood that the invention is not limited to such disclosed embodiments. Rather, the invention can be modified to incorporate any number of variations, alterations, substitutions or equivalent arrangements not heretofore described, but which are commensurate with the scope of the invention. Additionally, while various embodiments of the invention have been described, it is to be understood that aspects of the invention may include only some of the described embodiments. Accordingly, the invention is not to be seen as limited by the foregoing description, but is only limited by the scope of the appended claims.

1. An optical component comprising:
a substrate;
an input arranged to receive light;
an output arranged to output light; and
a waveguide arrangement arranged to direct light propagating in a transverse magnetic (TM) mode from the input to the output;
wherein said waveguide arrangement comprises a membrane extending over a channel in the substrate to form an unsupported portion that is separated from the substrate by 10 μm or more, said unsupported portion comprising an uninterrupted core section extending between first and second surfaces of the membrane and a structured section extending between the first and second surfaces adjacent the core section comprising a plurality of holes in said membrane, wherein the core section and the structured section define common planes at the first and second surfaces;
wherein the core and structured sections form a waveguide arranged to direct light at least partway from the input to the output;
wherein one or more parameters of the holes are chosen such that the TM guided mode in the core section falls below a dielectric band defined by the holes in the structured section; and
wherein the membrane comprises a thickness selected to minimize an internal confinement factor of the waveguide formed by the core and structured sections.

2. The optical component of claim 1, arranged such that the interaction of an evanescent field of light propagating in the waveguide with a surrounding medium exceeds an interaction possible with an equivalent length free-space beam.

3-5. (canceled)

6. The optical component of claim 1, wherein the waveguide arrangement is arranged to direct mid-infrared light from the input to the output.

7-8. (canceled)

9. The optical component of claim 1, wherein the membrane comprises a thickness between 50 nm and 1000 nm.

10-11. (canceled)

12. The optical component of claim 1, wherein the waveguide follows a path that contains one or more complete loops between the input and the output.

13. The optical component of claim 1, wherein the waveguide follows a clothoid curve path.

14. The optical component of claim 1, wherein the unsupported portion of the membrane has a width of 10 μm or more.

15. The optical component of claim 1, comprising a photonic chip.

16. The optical component of claim 1, comprising an integral sample chamber adjacent the unsupported portion of the membrane.

17. The optical component of claim 1, wherein the membrane is formed from Silicon Nitride (SiNₓ), Tantalum Oxide (Ta₂O₅), Aluminium Oxide (Al₂O₃), Silicon (Si), Germanium (Ge), Diamond (C), Indium Phosphide (InP), or Gallium Arsenide (GaAs).

18. The optical component of claim 1, wherein the holes may have a maximum dimension of 2 μm or less.

19. The optical component of claim 1, wherein the holes in the structured section are substantially circular.

20. The optical component of claim 1, wherein some of or all of the holes in the structured section form a periodic array such as a regular or irregular lattice.

21. (canceled)

22. The optical component of claim 1, wherein the holes have an average spacing that is less a half a target wavelength of light for which the waveguide arrangement is arranged.

23. A sensing system comprising:
a light source;
an optical component comprising:
a substrate;
an input arranged to receive light from the light source;
an output arranged to output light; and
a waveguide arrangement arranged to direct light propagating in a transverse magnetic (TM) mode from the input to the output;
wherein said waveguide arrangement comprises a membrane extending over a channel in the substrate to form an unsupported portion that is separated from the substrate by 10 μm or more, said unsupported portion comprising an uninterrupted core section extending between first and second surfaces of the membrane and a structured section extending between the first and second surfaces adjacent the core section comprising a plurality of holes in said membrane, wherein the core section and the structured section define common planes at the first and second surfaces;
wherein the core and structured sections form a waveguide arranged to direct light at least partway from the input to the output;
wherein one or more parameters of the holes are chosen such that the TM guided mode in thes core section falls below a dielectric band defined by the holes in the structured section; and
wherein the membrane comprises a thickness selected to minimize an internal confinement factor of the waveguide formed by the core and structured sections;
a sample volume adjacent the optical component; and
a detector arranged to receive light from the output of the optical component.

24. The sensing system of claim 23, wherein the sample volume has a volume of 100 ul or less.

25. A method of manufacturing an optical component comprising:
applying a membrane to a substrate; and
forming a waveguide arrangement using said membrane and said substrate, said waveguide arrangement arranged to direct light propagating in a transverse magnetic (TM) mode from an input arranged to receive light to an output arranged to output light;
wherein forming said waveguide arrangement comprises:
forming a plurality of holes in the membrane to form a structured section adjacent an uninterrupted core section, the structured and core sections extending between first and second surfaces of the membrane and defining common planes at the first and second surfaces, wherein one or more parameters of the holes are chosen such that the TM guided mode in the core section falls below a dielectric band defined by the holes in the structured section;
under-etching the substrate through the holes in the membrane to form a channel in the substrate aligned with the structured section and the uninterrupted core section, such that the core and structured sections are comprised by an unsupported portion of the membrane that is separated from the substrate by 10 μm or more and such that the core and structured sections form a waveguide arranged to direct light at least partway from the input to the output, wherein the membrane comprises a thickness selected to minimize an internal confinement factor of the waveguide formed by the core and structured sections.

26. The optical component of claim 1, wherein the core section does not protrude relative to the structured section.

27. The optical component of claim 1, wherein the one or more parameters of the holes comprises an average spacing of the holes.

28. The optical component of claim 1, wherein the one or more parameters of the holes comprises a filling factor of the holes.
